(19) United States
(12) Patent Application Publication
(10) Pub. No.: US 2007/0237828 A1
(43) Pub. Date: Oct. 11, 2007

(54) Ziprasidone Dosage Form

(75) Inventors: Gouri Shankar Vibhuthi, Hauppauge, NY (US); Sudeep Kumar Agrawal, Hyderabad (IN); Billa Praveen Reddy, Hyderabad (IN); Kiran Krishnan, Palatine, IL (US); Mailatur Sivaraman Mohan, Hyderabad (IN)

Correspondence Address: DR. REDDY'S LABORATORIES, INC., 200 SOMERSET CORPORATE BLVD, SEVENTH FLOOR,, BRIDGEWATER, NJ, 08807-2862, US

(73) Assignees: DR. REDDY'S LABORATORIES LIMITED, HYDERABAD, AND HREA PRADESH INDIA (IN); DR. REDDYS LABORATORIES, INC., BRIDGEWATER, NEW JERSEY, NJ (US)

(21) Appl. No.: 11/570,392

(22) PCT Filed: Jun. 9, 2005
(86) PCT No.: PCT/US05/20417

§ 371 Date: Dec. 11, 2006

(30) Foreign Application Priority Data
Jun. 11, 2004 (IN) 546/CHE/2004

(51) Int. Cl. A61K 9/14

(52) U.S. Cl. 424/489

(57) ABSTRACT

Pharmaceutical formulations of ziprasidone comprise ziprasidone or a salt thereof, in the form of particles having a mean particle size greater than about 90 μm and a pharmaceutically acceptable excipient.

This invention relates to a dosage form comprising ziprasidone or a salt thereof and a pharmaceutically acceptable excipient or a combination of excipients. One embodiment of the composition comprises ziprasidone hydrochloride particles having a mean particle size greater than 90 μm and a pharmaceutically acceptable excipient or a combination of excipients.

The drug ziprasidone has shown utility as a psychotropic agent, for treating schizophrenia and bipolar mania, and is commercially used in the form of ziprasidone hydrochloride monohydrate, having the chemical name 5-[2-[4-(1,2-benzisothiazol-3-yl)-1-piperazinyl]ethyl]-6-chloro-1,3-dihydro-2H-indol-2-one, monohydrochloride, monohydrate, and the empirical formula C₂₁H₂₁ClN₄OS.HCl.H₂O. The commercial product has the name GEODON™ and is available in capsules for oral dosing that contain 20, 40, 60, and 80 mg of the drug.

Low-solubility drugs often show poor bioavailability or irregular absorption, the degree of irregularity being affected by factors such as dose level, fed state of the patient, and form of the drug. Increasing the bioavailability of low-solubility drugs has been the subject of much research. Typical approaches can involve: (1) using particular formulation excipients, which increase solubility, for example surfactants; and/or (2) formulating the drug in a small particle size, thereby increasing the surface area of the drug to facilitate more rapid dissolution. Manipulating the particle size can present technical difficulties and expensive formulation and quality control challenges.

It is disclosed in U.S. Pat. Nos. 4,831,031 and 5,312,925 that ziprasidone is typically administered as the hydrochloric acid addition salt. The hydrochloride salt is advantageous in that it is a high permeability drug, a factor that favorably affects bioavailability. The hydrochloride salt does, however, posses relatively poor aqueous solubility, a factor that unfavorably affects bioavailability.

Increasing drug solubilization by using combinations of drug and polymer has been disclosed. For example Martin et al., in U.S. Pat. No. 4,344,934 mixed poorly-soluble drugs with polymers such as hydroxypropyl methylcellulose (HPMC) and added an aqueous surfactant solution to the drug-polymer mixture. While this results in improved dissolution, there is only slight enhancement of drug concentration in plasma, relative to the equilibrium concentration.

U.S. Pat. No. 5,955,459 describes the covalent conjugates of a fatty acid with certain antipsychotic agents, giving the unexpected property of extended therapeutic effectiveness.

Yet another technique for temporarily achieving a greater than equilibrium concentration of drug in a use environment is to formulate the drug as an aqueous or organic solution. For example, drug can be dissolved in polyethylene glycol (PEG) or an aqueous solution of PEG to which an acid or base may be added, or the drug can be dissolved in an aqueous solution of an acid or base. Alternatively, the drug can be dissolved in a pharmaceutically acceptable organic liquid such as glycerol, mono-, di-, or triglycerides, fats or oils.

While these solubility-improved drug forms show initially enhanced concentration of the drug in a use environment, nevertheless the improved concentration is often short-lived. Typically, the initially enhanced drug concentration is only temporary and quickly returns to the lower equilibrium concentration. For example, while a particular salt form of a basic drug may show improved initial aqueous concentration, the drug often rapidly converts in gastric fluid to another salt form (typically the HCl salt form) that has a much lower equilibrium concentration. In other cases, the drug maintains acceptable solubility in the low pH gastric solution, but precipitates, typically as the free-base form of the drug, upon passing into the small intestine where the pH is higher, typically from 4.4 to 7.5. Since drug absorption occurs primarily in the intestines, such drug dosage forms that do not sustain high concentration of the drug in an intestinal solution typically yield only minor improvements in bioavailability. Likewise, a high-solubility salt form of an acidic drug can rapidly convert to another salt form that has a much lower equilibrium concentration. Similar effects are observed even for high solubility salt forms of zwitterionic drugs. Similarly, once the high-energy crystalline form of a drug (e.g., a polymorph) dissolves, the drug often rapidly precipitates or crystallizes from solution as it changes to a lower energy crystalline form or an amorphous form with lower solubility which causes concentration of dissolved drug to approach a lower equilibrium concentration.

U.S. Pat. No. 6,150,366 describes compositions comprising crystalline ziprasidone free base or crystalline ziprasidone hydrochloride particles having a mean particle size equal to or less than 85 μm and a pharmaceutically acceptable carrier, as being substantially bioequivalent and useful to treat psychoses such as schizophrenia. The term “bioequivalent” in the patent means that the pharmacokinetic parameter “AUC” of a dosage form is not less than 80 percent of the value obtained from dosing an equivalent formulation having ziprasidone present in a mean particle size of 20 μm. The patent also discloses that ziprasidone hydrochloride dissolution rate in aqueous media, at least at or below 85 μm, does not vary substantially with particle size and therefore appears to be largely independent of size. However, the patent states that the reduced particle size approach to enhance the bioavailability of a drug can present difficult and expensive formulation and quality control challenges.

U.S. Pat. No. 6,399,777 teaches improvements to the solubility of ziprasidone hydrochloride salts by preparing inclusion complexes of ziprasidone hydrochloride. The patent teaches the use of cyclodextrin to form complexes with the poorly soluble drugs to improve the bioavailability of the drugs. Cyclodextrin preparations have several disadvantages, as the drug loading is low and this method only works with drugs which fit into the cavity of the cyclodextrin and which have a high complex-forming constant. The molecular structure, the polarity, the size and the possibility for interactions with the cyclodextrin molecule are important factors determining the success of cyclodextrin-preparations.

One approach to increase the bioavailability of low-solubility drugs has involved forming amorphous dispersions of drugs with polymers. Examples of attempts to increase drug bioavailability by forming a dispersion of the drug with a polymer are discussed in U.S. Patent Application Nos. US 2003/0228358, US 2003/0224043 and US 2003/0219489.

However, creating an amorphous dispersion of a drug and polymer does have some drawbacks. There is a risk that in the process of creating the dispersion, the drug will be altered. For example, ziprasidone hydrochloride may degrade at the elevated temperatures used to form some dispersions. Some processes use organic solvents, which must be thoroughly removed to avoid drug degradation. Solvents must be chosen which dissolve both the drug and the polymer. The process of forming such dispersions is also time-consuming and expensive. In addition, the dispersions may in some cases be unstable and may either chemically degrade over time at moderate temperature and humidity levels or the drug may convert to a lower energy and lower solubility polymorphic form.

All of the above techniques used to make a formulation of ziprasidone hydrochloride use a technique which is time consuming and involves numerous complexities as a part of the process, and not all the process are economical. Hence, there is a need for a process to formulate ziprasidone hydrochloride wherein the pharmacological characteristics are independent of the particle size of ziprasidone hydrochloride and produce the requisite plasma concentrations for the desired pharmacological effect.

### SUMMARY OF THE INVENTION

In one aspect, the invention provides a dosage form comprising ziprasidone or a salt thereof in the form of particles having a mean size at least about 90 μm, and having a ziprasidone bioavailability equal to or greater than the bioavailability of a dosage form where ziprasidone or a salt thereof is present as particles having a mean size less than 85 μm.

In another aspect, the invention provides a solid dosage form comprising ziprasidone hydrochloride particles having a mean size at least about 90 μm and a hydrophilic excipient.

In a further aspect, the invention provides a solid dosage form comprising ziprasidone hydrochloride particles having a mean size between about 90 and about 180 μm, at least about 90 volume percent of the particles having sizes no greater than about 220 μm, and a sorbitan derivative surfactant in an amount that is about 0.05 to about 5 weight percent of the total dosage form.

These and other aspects of the invention will be apparent from the description and examples that follow.

### DETAILED DESCRIPTION

The present invention is based on the surprising observation that formulations comprising solid ziprasidone hydrochloride having mean particle sizes greater than about 90 μm can be used to prepare therapeutically useful dosage forms. Accordingly, the invention provides a pharmaceutical composition comprising ziprasidone free base or ziprasidone hydrochloride particles having a mean particle size greater than about 90 μm as measured by the Malvern light scattering technique, together with one or more pharmaceutical excipients and a pharmaceutically acceptable diluent or carrier.

It is frequently desired that the ziprasidone hydrochloride particles in the compositions of the invention have a D₉₀ not exceeding about 220 μm. The notation D_{X} means that X volume percent of the particles have a diameter less than or equal to the specified diameter, as measured by the Malvern light scattering technique. Thus, D₉₀≦220 μm means that 90 volume percent of the particles have a diameter that does not exceed 220 μm. The terms “size” and “diameter” will be used herein interchangeably, it being recognized that particles can have different shapes where the term “diameter” might not strictly apply.

The range of mean particle sizes for use in the invention will usually be 90 to 180 μm, or 120 to 150 μm, or 130 to 140 μm.

The formulations of this invention are advantageous because the useful ziprasidone hydrochloride drug particles can have a wide range of particle sizes. In line with the invention, particle size no longer remains a critical constraint in development of a dosage form of ziprasidone hydrochloride. Thus, ziprasidone free base, ziprasidone hydrochloride, or another ziprasidone salt can be successfully formulated even though the drug substance has an average particle size greater than about 90 μm.

It will be appreciated by those skilled in the art of powder production that there are numerous known size reduction methods, which can be applied for producing crystalline ziprasidone hydrochloride particles having a mean particle size equal to or less than about 85 μm. However, all these methods involve many complexities and need a very stringent control to produce the desired sizes and distributions. Ziprasidone and its salts can be produced in a manner that directly forms crystals having appropriate sizes for use in the formulations of the present invention, so that there is no need for further size reduction procedures.

By incorporating hydrophilic excipients in the pharmaceutical compositions, the aqueous solubility of ziprasidone and its salts is increased. The hydrophilic excipients are thought to act by decreasing surface tension and thereby forming micelles that assist in the solubilization of ziprasidone or the salt having particle sizes greater than about 90 μm. Further, the hydrophilic excipient is believed to facilitate transport of the dissolved drug through cellular membranes into tissues. However, this invention is not to be considered as being bound to these or any other theories of operation, as other factors could be relevant.

Among the useful hydrophilic excipients that are suitable for use in the invention are surface active agents. In particular, nonionic surfactants have been found useful. Many available useful nonionic surfactants are sorbitan derivatives, e.g., sorbitan ethers or sorbitan esters.

Polysorbate surfactants are mixtures of sorbitol and sorbitol anhydride fatty acid esters, which have been condensed with ethylene oxide. A large number of commercial polysorbate surfactants can be used in the present invention, including the following:

| Polysorbate | Chamical Name |
|---|---|
| 20 | Polyoxyethylene 20 sorbitan monolaurate |
| 21 | Polyoxyethylene (4) sorbitan monolaurate |
| 40 | Polyoxyethylene 20 sorbitan monopalmitate |
| 60 | Polyoxyethylene 20 sorbitan monostearate |
| 61 | Polyoxyethylene (4) sorbitan monostearate |
| 65 | Polyoxyethylene 20 sorbitan tristearate |
| 80 | Polyoxyethylene 20 sorbitan monooleate |
| 81 | Polyoxyethylene (5) sorbitan monooleate |
| 85 | Polyoxyethylene 20 sorbitan trioleate |

When the hydrophilic excipient is a surfactant or a mixture of two or more surfactants, this component will be present in the compositions of the present invention in amounts about 0.05 to about 5 percent by weight of the final dosage form, or about 0.1 percent to about 2 percent, or about 0.2 percent to about 0.6 percent. The amount of surfactant in the dosage form can be adjusted to provide different ziprasidone bioavailability parameters; increasing the surfactant concentration generally results in an increased drug bioavailability. The amount of surfactant also can be adjusted to compensate for larger or smaller drug particle sizes, as larger particles can require the use of higher surfactant concentrations to achieve a target bioavailability.

The concept of bioavailability as contemplated herein is in accordance with industry-accepted standards. Bioavailability is indicated by integrating the plasma concentrations of a drug substance at various times after dosing to give an “area under the curve,” or “AUC,” beginning at the time of dosing (time=0) and ending at either infinite time or the time (denoted “t”) at which the drug concentration in the plasma becomes unmeasurable. Bioavailability is also indicated by determining the elapsed time after dosing at which plasma concentration of the drug is highest (“Tₘₐₓ”) and that actual maximum plasma concentration (“Cₘₐₓ”). “Bioequivalence” is indicated in accordance with applicable regulatory standards, and in many countries is established when bioavailability parameters for one dosage form of a drug are at least 80 percent, but not more than 125%, of the same parameters for another dosage form.

In addition to the drug and hydrophilic excipient, depending on the selected embodiment of the dosage form, there will also be included one or more components chosen from one or more of diluents, disintegrants, binders, glidants, and lubricants. Useful diluents include: mono- di-, and polysaccharides such as dextrose, lactose, maltodextrin, trehalose, and maltose; mannitol and sorbitol; celluloses such as microcrystalline cellulose, powdered cellulose, and microfine cellulose; and starches, including starch derivatives. Binders include: acacia; alginic acid; carbomers; sodium carboxymethylcellulose; dextrin; guar gum; hydroxypropyl methylcellulose; glucose; maltodextrin, methylcellulose; polymethacrylates; polyvinylpyrrolidones (“povidones”); pregelatinized starch; sodium alginate; and starch. Disintegrants include: alginic acid; sodium carboxymethylcellulose; microcrystalline cellulose; polacrylin potassium; sodium alginate; sodium starch glycolate; and starch. These lists are not intended to be complete, as many other substances and classes of excipients are available for use in formulating solid dosage forms, and their utility in the present invention will be appreciated by those skilled in the art.

In a typical embodiment of the invention, a diluent will comprise about 25 to about 65 percent by weight of the dosage form, and binder will comprise about 0.25 to about 10 percent by weight of the final dosage form. The actual amounts, however, can vary widely for different formulation types and will not necessarily fall within these ranges.

Dosage forms of the invention can be produced using any of the customary procedures. These include simply mixing the desired components, then filling the mixtures into capsules or compressing the mixtures into tablets of a desired size and shape. Alternatively, the component mixtures can be granulated, either in the dry form or using a binder solution, then dried (if necessary) and filled into capsules or compressed into tablets. Those skilled in the art can easily develop a convenient procedure for producing a desired dosage form, with operational steps that are very commonly used.

The invention will be further explained by means of the following examples, which are provided only to illustrate certain aspects of the invention and are not intended to limit the scope of the claimed invention.

### EXAMPLE 1

Capsules containing 80 mg of ziprasidone were prepared using the following:

| Ingredient | mg/Capsule |
|---|---|
| Ziprasidone * | 80 |
| Lactose anhydrous | 167.6 |
| Pregelatanized starch | 36 |
| Polysorbate 80 | 1.5 |
| Povidone K-30 | 3 |
| Isopropyl alcohol | q.s. |
| Magnesium stearate | 1.8 |
| Silicon dioxide | 3 |

* Ziprasidone was contained in ziprasidone hydrochloride.

Ziprasidone hydrochloride, anhydrous lactose, and pregelatinized starch were sifted through a 40 mesh sieve and mixed for 5 minutes. Povidone K-30 was dissolved in isopropyl alcohol and to the solution polysorbate 80 was added and mixed. This solution was used for granulation of the dry blend and wet granules were sifted through a 10 mesh sieve and dried at 70±5° C. for one hour. Dried granules were sifted through a 16 mesh sieve and a mixture of magnesium stearate and silicone dioxide that had been sifted through a 40 mesh sieve was added to the granules and mixed for 5 minutes. This lubricated blend was filled into size 2 hard gelatin capsules. Temperature and humidity conditions maintained during processing were 25.5° C. and 52% RH.

### EXAMPLE 2

A two way crossover clinical study was conducted involving 8 human subjects. The subjects were dosed with ziprasidone hydrochloride 80 mg capsules having drug particle sizes of 10-40 μm (as the GEODON marketed formulation of ziprasidone hydrochloride) and with ziprasidone hydrochloride 80 mg capsules, prepared according to the procedure of Example 1 and using drug substance having a mean particle size of 100-150 μm. The study produced the following results:

| PHARMACOKINETIC | EXAMPLE 1 | | GEODON | |
|---|---|---|---|---|
| PARAMETER | PRODUCT | | 80 mg | |
| Mean AUC₀₋ₜ | 738.6 | ng · hr/ml | 489.06 | ng · hr/ml |
| Mean AUC_{0-∞} | 801.16 | ng · hr/ml | 527.9 | ng · hr/ml |
| Mean Cₘₐₓ | 78.63 | ng/ml | 59.3 | ng/ml |
| Mean Tₘₐₓ | 4.5 | hours | 4.5 | hours |

The above pharmacokinetic data indicate that the invention product has an enhanced bioavailability, as compared to the GEODON marketed formulation of ziprasidone hydrochloride. The above data show the profound impact of hydrophilic excipients on bioavailability of the ziprasidone hydrochloride.

### EXAMPLE 3

Capsules containing either 20, 40, 60, or 80 mg of ziprasidone were prepared using the following components:

| | |
|---|---|
| Ingredient | mg/Capsule |

Dry Mixture

| | | | | |
|---|---|---|---|---|
| Ziprasidone * | 20 | 40 | 60 | 80 |
| Anhydrous lactose | 36.2 | 72.4 | 108.6 | 144.8 |
| Pregelatinized starch | 9 | 18 | 27 | 36 |
| Silicon dioxide | 0.75 | 1.5 | 2.25 | 3 |

Granulating Solution

| | | | | |
|---|---|---|---|---|
| Povidone K-30 | 0.75 | 1.5 | 2.25 | 3 |
| Polysorbate 80 | 0.175 | 0.35 | 0.525 | 0.7 |
| Isopropyl alcohol | q.s. | q.s. | q.s. | q.s. |

Lubricant

| | | | | |
|---|---|---|---|---|
| Magnesium stearate | 0.6 | 1.2 | 1.8 | 2.4 |
| Silicon dioxide | 0.75 | 1.5 | 2.25 | 3 |

* Ziprasidone was contained in ziprasidone hydrochloride.

The starch was Starch 1500 LM, sold by Colorcon of West Point, Pa. U.S.A. This material has a moisture content less than 7 weight percent.

The silicon dioxide was micronized material sold as SYLOID™ AL 1-FP by Grace Davison, W.R. Grace & Co., Columbia, Md. U.S.A.

The drug substance was a combination of three batches of ziprasidone hydrochloride having the following particle sizes:

| Batch | D₉₀ (μm) | Mean Particle Size (μm) |
|---|---|---|
| 1 | 231 | 147 |
| 2 | 244 | 154 |
| 3 | 231 | 129 |

Ziprasidone hydrochloride, lactose, starch and silicon dioxide were sifted together through a 40 mesh sieve and mixed for 15 minutes in a rapid mixer granulator. Povidone and polysorbate 80 were dissolved in isopropyl alcohol and added to the dry mixture in the granulator, then the granulated mass was dried and sifted through a 20 mesh sieve. Retained particles were recovered from the sieve, milled through a 1.5 mm sieve, passed through the 20 mesh sieve, and combined with the other granules. Magnesium stearate and the second portion of silicon dioxide were sifted together through a 60 mesh sieve and blended with the granules in a double cone blender. Quantities of lubricated granules were measured into hard gelatin capsules of size 4 (for 20 and 40 mg doses), size 3 (for 60 mg doses), or size 2 (for 80 mg doses).

As an alternative to the capsule dosage form, the lubricated granules can be compressed into tablets, using customary tableting equipment and appropriately sized and shaped punches and dies for the desired doses.

### EXAMPLE 4

A two way crossover clinical study was conducted in a manner similar to that of Example 2, using the 20 mg dosage forms prepared in Example 3 and 47 fasted human subjects. The study produced the following results:

| PHARMACOKINETIC | EXAMPLE 3 | | GEODON | |
|---|---|---|---|---|
| PARAMETER | PRODUCT | | 20 mg | |
| Mean AUC₀₋ₜ | 209.21 ± 81.01 | ng · hr/ml | 215.21 ± 79.32 | ng · hr/ml |
| Mean AUC_{0-∞} | 223.53 ± 83.47 | ng · hr/ml | 225.69 ± 80.10 | ng · hr/ml |
| Mean Cₘₐₓ | 28.25 ± 12.45 | ng/ml | 30.93 ± 13.49 | ng/ml |
| Mean Tₘₐₓ | 4.11 ± 1.19 | hours | 4.50 ± 1.26 | hours |

These results indicate bioequivalence of the Example 3 product and the GEODON capsules.

### EXAMPLE 5

A two way crossover clinical study was conducted in a manner similar to that of Example 2, using the 20 mg dosage forms prepared in Example 3 and 35 fed human subjects. The study produced the following results:

| PHARMACOKINETIC | EXAMPLE 3 | | GEODON | |
|---|---|---|---|---|
| PARAMETER | PRODUCT | | 20 mg | |
| Mean AUC₀₋ₜ | 548.15 ± 176.09 | ng · hr/ml | 545.17 ± 196.13 | ng · hr/ml |
| Mean AUC_{0-∞} | 555.63 ± 179.02 | ng · hr/ml | 555.50 ± 200.05 | ng · hr/ml |
| Mean Cₘₐₓ | 64.73 ± 21.42 | ng/ml | 61.99 ± 26.25 | ng/ml |
| Mean Tₘₐₓ | 6.89 ± 1.82 | hours | 7.20 ± 2.47 | hours |

These results indicate bioequivalence of the Example 3 product and the GEODON capsules.

1. A dosage form comprising ziprasidone or a salt thereof in the form of particles having a mean size at least about 90 μm, and a hydrophilic excipient.

2. The dosage form of claim 1, wherein ziprasidone is present as ziprasidone hydrochloride.

3. The dosage form of claim 1, wherein particles of ziprasidone or a salt thereof have a mean size between about 90 and about 180 μm.

4. The dosage form of claim 1, wherein at least about 90 volume percent of the particles of ziprasidone or a salt thereof have sizes no greater than about 220 μm.

5. (canceled)

6. The dosage form of claim 1, comprising a surfactant.

7. The dosage form of claim 1, comprising a sorbitan derivative surfactant.

8. The dosage form of claim 1, comprising a polysorbate surfactant in an amount that is about 0.05 to about 5 weight percent of the total dosage form.

9. The dosage form of claim 1, wherein particles of ziprasidone or a salt thereof have a mean size between about 90 and about 180 μm and the dosage form comprises a surfactant.

10. The dosage form of claim 1, wherein particles of ziprasidone or a salt thereof have a mean size between about 90 and about 180 μm and the dosage form comprises a sorbitan derivative surfactant.

11. The dosage form of claim 1, wherein particles of ziprasidone or a salt thereof have a mean size between about 90 and about 180 μm and the dosage form comprises a sorbitan derivative surfactant in an amount that is about 0.05 to about 5 weight percent of the total dosage form.

12. The dosage form of claim 1, wherein particles of ziprasidone or a salt thereof have a mean size between about 90 and about 180 μm and at least about 90 volume percent of the particles of ziprasidone or a salt thereof have sizes no greater than about 220 μm.

13. A solid dosage form comprising ziprasidone hydrochloride particles having a mean size at least about 90 μm and a hydrophilic excipient.

14. The solid dosage form of claim 13 wherein ziprasidone hydrochloride particles have a mean size between about 90 and about 180 μm.

15. The solid dosage form of claim 13, wherein the hydrophilic excipient comprises a surfactant.

16. The solid dosage form of claim 13, wherein the hydrophilic excipient comprises a sorbitan derivative surfactant.

17. The solid dosage form of claim 13, wherein particles of ziprasidone hydrochloride have a mean size between about 90 and about 180 μm and the dosage form comprises a polysorbate surfactant in an amount that is about 0.05 to about 5 weight percent of the total dosage form.

18. A solid dosage form comprising ziprasidone hydrochloride particles having a mean size between about 90 and about 180 μm, at least about 90 volume percent of the particles having sizes no greater than about 220 μm, and a sorbitan derivative surfactant in an amount that is about 0.05 to about 5 weight percent of the total dosage form.

19. The solid dosage form of claim 18, wherein the surfactant comprises a polysorbate.

20. The solid dosage form of claim 18, wherein the surfactant comprises about 0.1 percent to about 2 percent of the dosage form.
